(19) United States
(12) Patent Application Publication
(10) Pub. No.: US 2014/0356343 A1
(43) Pub. Date: Dec. 4, 2014

(54) COMPOSITION FOR TREATING PARKINSON'S DISEASE

(71) Applicant: KEY NEUROSCIENCES SAS, Paris (FR)

(72) Inventors: Thomas N. Chase, Washington, DC (US); Justin D. Oh-Lee, Mount Pleasant, MI (US)

(21) Appl. No.: 14/362,666

(22) PCT Filed: Dec. 4, 2012
(86) PCT No.: PCT/EP2012/074386

§ 371 Date: Jun. 4, 2014

Related U.S. Application Data
(60) Provisional application No. 61/567,105, filed on Dec. 5, 2011.

(51) Int. Cl. A61K 38/12; A61K 38/47; A61K 38/46; A61K 38/17

(52) U.S. Cl. CPC A61K 38/12; A61K 38/1767; A61K 38/47; A61K 38/465
USPC 424/94.62; 514/17.7; 424/94.6

(57) ABSTRACT

A composition useful for the treatment of degenerative brain disease. A pharmaceutical composition comprising an effective amount of apamin as an active ingredient and an effective amount of at least another compound including a permeability enhancer and/or carrier molecule.

The present invention relates to a composition useful for the treatment of degenerative brain diseases. More particularly, the invention relates to a pharmaceutical composition comprising apamin as an active ingredient and at least another compound for the treatment of Parkinson's disease and related parkinsonian disorders.

In the twentieth century, as the average life span of human has been increasing with the rapid development of medicine, new social problems including increased population ratio of older people are coming to the front, especially, the geriatric neuronal diseases such as stroke, Alzheimer's disease (AD), Parkinson's disease (PD) etc., which are fatal functional disorder of neuronal system, have been increased.

Especially, PD is caused by the death of substantia nigra pars compacta neurons in brain and is a frequently occurring neurological disease. In particular, idiopathic PD predominate over 80% among the people suffering from PD.

PD is a progressive neurodegenerative disorder characterized by the loss of the nigrostriatal pathway. Although the cause of PD is not known, it is largely associated with the progressive death of dopaminergic (tyrosine hydroxylase (TH) positive) mesencephalic neurons, inducing motor impairment. Symptoms of PD include hypokinesia (reduction in movement), bradykinesia (slowness of movement), rigidity, postural instability and rest tremors. Although PD is predominantly a movement disorder, other impairments frequently develop, including psychiatric issues such as depression and dementia, Autonomic disturbances and pain can occur in later stages, and as PD progresses, it causes significant disability and impaired quality of :life for the affected person.□□

A therapeutic strategy currently in use for treating PD consists of dopaminergic replacement. Symptomatic treatment of the disease-associated motor impairments involves oral administration of the dopamine precursor dihydroxyphenylalanine (L-Dopa). In early stage PD, L.-Dopa is efficacious, but patients progressively lose the ability to convert L-Dopa to dopamine as more and more dopaminergic neurons degenerate. In addition, after long-term L-Dopa therapy, L-Dopa treatment begins to cause severe side effects, including drug-induced dyskinesias.□

Another strategy for the treatment of PD is gene therapy. Viral vector-based approaches are being evaluated for the treatment of various neurological diseases, through the introduction of therapeutic genes by transduction of the viral vector into neuronal and/or support cells. This approach has not yet shown a radical efficacy and is often difficult to reduce to practice.

There is currently no satisfactory cure for PD.

The use of honeybee venom (apitoxin) for the treatment of PD has been recently described. It can be mentioned WO2009/022067 which describes the use of bee venom, in a single dose, for the treatment of PD. Apitoxin consists of at least 18 pharmacologically active components. The ratio of the main components in Apitoxin are: Phospholipase A2 (10-12%), Melittin (40-50%), Apamin (3%), Histamine (0.66-1.6%), Dopamine (0.13-1%) and Noradrenaline (0.1-0.7%). In the same way, WO2010/134476 also describes different technics to obtain purified bee venom for use in the treatment of PD.

The systemic administration (by ip or sc injection) of honeybee venom (apitoxin) unexpectedly induces, over a broad range of doses (approximately 70 to 700 μg/300 gm rat) and for a prolonged period (at least 12 hours), both contralateral and ipsilateral rotations in the standard 6-OHDA rat model of PD. There is no observable evidence that apitoxin exerts other concomitant actions. Surprisingly, these observations as presented below, indicate that apitoxin affects motor behavior in this animal model not by the pharmacologic action of a single component but rather by a unique synergistic response to several of its constituents, acting by a variety of mechanisms. The implication of these findings is that apitoxin and/or its constituents, alone or especially in combination, should be far more beneficial in the treatment of human PD than hitherto imagined.

The simultaneous tendency of rats unilaterally lesioned with 6-OHDA to turn in both a direction ipsilateral to their brain injury and in a direction contralateral to their brain injury following apitoxin administration is totally unexpected. Numerous studies in this animal model over several decades have documented that drugs, capable of producing an antiparkinsonian response in humans with PD, typically induce rotational activity either in the contralateral (as for example dopamine precursors such as levodopa or dopamine agonists such as bromocriptine and pramipexole) or in the ipsilateral direction (as for example amphetamine), but not in both directions concurrently. In view of the surprising dual action of apitoxin, it appears that it may possess an extraordinary ability to act at the same time on both the lesioned and on the intact nigrostriatal and downstream neuronal systems to influence motor behavior. For this reason, the therapeutic efficacy of apamin in PD patients should be greater than that of any drug now used in the treatment of this disease.

Another recent strategy, such as described in WO2009/022068, consists of using 1 to 10 micrograms of apamin in a single dose injection.

Unexpectedly, apamin was found to account for the motor response to apitoxin observed in the rat 6-OHDA PD model. At ip or sc doses between about 1 and 2 mg/kg, apamin induced a mixture of robust turning responses in both the ispilateral and contralateral directions lasting up to at least 12 hours. In all respects, apitoxin and apamin induced rotations appeared to be phenomologically similar.

In contrast, but equally surprising, it was found that none of the other major, currently identified, apitoxin components studied (including melittin, phospholipase A2, mast cell degranulating peptide, histamine, hyaluronidase, tertiapin, secapin, or HTGAVLAGV) possessed a similar ability to induce a conspicuous turning response in the 6-OHDA rat model. Thus it can be concluded that apamin uniquely accounts for most, if not all, the anti-parkinsonian-like activity of apitoxin in the rat 6-OHDA model.

No less surprising, apamine was observed to induce a variety of other, atypical motor responses. These included mixtures of bilateral and widely distributed dystonic and/or athetotic postures and motor activities, tremorous and shaking movements, as well as ataxic and other disorders of locomotion. All appeared dose dependent, with onset and duration similar to that of the rotational response. All could have profound implications on the utility of apamin alone for the treatment of PD.

Other unexpected characteristics of the motoric (antiparkinsonian-like) response to apamin in the rat 6-OHDA model could also materially influence its safety and efficacy in the treatment of PD. These include a substantially higher dose requirement, a steeper dose response relation, and a lower therapeutic index than apitoxin. Any of these factors, alone or in combination, could adversely affect the usefulness of apamin as a monotherapy for those suffering from PD and related parkinsonian disorders.

The motoric effects (turning behavior) of apamin in the rat 6-OHDA model have been attributed to its potent ability to act in the central nervous system as an irreversible inhibitor of SK channels, especially those of the SK-2 subtype. These channels are abundantly expressed throughout the brain, including regions controlling motor behavior, most particularly, those linked to the motor dysfunction associated with PD. Accordingly, the fact that apamin influences motor behaviors in animal models of PD is hardly surprising. But the characteristics of these responses, especially those characteristics that could materially affect the usefulness of apamin by itself as a treatment for PD, could hardly have been anticipated. It has been discovered, for example, that the dose of apamin required for a rotatory response approximating that of apitoxin in the 6-OHDA model is some 2 orders of magnitude (about 200 fold) greater than expected based on the concentration of apamin in apitoxin.

The present invention intends to remedy these drawbacks.

It is demonstrated in the present specification that other constituent(s) of apitoxin augment or potentiate the effects of apamin on motor behavior by increasing the availability of apamin to its sites of action in brain or by exerting a pharmacologic effect on brain function or both.

In the present specification, a novel and surprising effect of certain constituents of apitoxin is described. Such constituents, as it will be described below, do not present alone a significative activity but they present a synergetic activity when they are combined with apamin.

As it will be described in the present specification, data in support of this possibility has been found with melittin, phospholipase A2 (PLA-2) and hyaluronidase. All these constituents potentiated the rotatory response to apamin (melittin [such as observed with 0.25 mg/kg apamin and 0.75 mg/kg melittin] or PLA-2 [such as observed with 0.5 mg/kg apamin and 5 mg/kg PLA-2]) and/or reduced the atypical motor behaviors induced by apamin (as for example seen with 1.0 mg/kg apamin and 20 mg/kg hyaluronidase). All these apitoxin components are permeability enhancers that could improve the availability of apamin to its central sites of action. Other apitoxin components may be identified to have similar “carrier” actions. Some of these carrier molecules (for example melittin or PLA-2) also significantly hasten the onset of rotational action of apamin, presumably attributable to their permeability enhancement capabilities.

In conclusion, apitoxin contains a complex mixture of pharmacologically active substances of potential relevance to the treatment of PD. Some, especially apamin, act at SK channels in the basal ganglia and possibly elsewhere to influence motor function. Others, including hyaluronidase, act as carrier molecules that modify the transport and distribution of the channel-active constituents. It follows that while apamin alone may find limited utility in the treatment of PD for the reasons presented above, apamin in combination with certain other apitoxin components likely explains the reported success of apitoxin in a single patient with PD and (based on our unanticipated rat 6-OHDA model results) should confer substantial benefit to patients with this and related disorders compared to any currently available medication. These benefits include a greater degree of symptom relief and at all stages of disease, a faster onset and longer duration of action, and a reduced tendency to induce the motor complications of the type that now disable most late stage PD patients.

Generally speaking, the invention relates to a pharmaceutical composition comprising an effective amount of apamin as an active ingredient and an effective amount of at least another compound acting as a potentiator of the apamin activity.

The term “potentiator” must be understood in general terms as a compound that enhance, ameliorate or improve the activity of the apamin by any mechanism of action. Without being linked by a theory, it is believed that the said potentiator can act as a permeability enhancer and/or carrier molecule.

In a first embodiment the invention relates to a pharmaceutical composition comprising an effective amount of apamin as an active ingredient and an effective amount of at least another compound consisting of a permeability enhancer and/or carrier molecule.

By the expression “effective amount”, it is intended a dosage sufficient to produce a desired result on a health condition, pathology, and disease of a subject or for a diagnostic purpose. The desired result may comprise a subjective or objective improvement in the recipient of the dosage.

Apamin is well known by the person skilled in the art. Apamin can consist of natural apamin derived from various species and, more preferably, honey bee. Apamin can also be chemically or biologically synthetized by any method known in the art. It is also mentioned here that any analog or derivative known by the said person skilled in the art can be used in replacement or in addition with the apamin.

According to the invention, any permeability enhancer can be used in combination with apamin. By the expression “permeability enhancer”, it is intended any compound that could improve the availability of apamin to its central sites of action.

According to the invention, any carrier molecule can be used in combination with apamin. By the expression “carrier molecule”, it is intended any compound that modify the transport and distribution of the channel-active constituents.

In another preferable aspect the present invention consists in a pharmaceutical composition comprising i) a compound such as, without limitation, apamin, acting at SK channels in the basal ganglia and possibly elsewhere to influence motor function, and ii) at least another compound, such as without limitation Hyalurinidase, PL-A2, and melittin acting as a carrier molecules that modify the transport and distribution of the channel-active constituents.

The present invention consists also in a pharmaceutical composition comprising i) a compound such as, without limitation, apamin, acting at SK channels in the basal ganglia and possibly elsewhere to influence motor function, and ii) at least another compound, such as without limitation Hyalurinidase, PL-A2, and melittin acting as permeability enhancers that could improve the availability of apamin to its central sites of action. The invention relates also to the use of the pharmaceutical composition above described for the treatment of PD and related parkinsonian disorders.

A particular innovative aspect according to the invention relates to the selection of the combined compounds to apamin. There is no teaching in the prior art suggesting to select melittin, and/or hyaluronidase and/or phospholipase A-2 (compared to all the constituent of apitoxin) as of interest in combination with apamin. Moreover, regarding the ratio of these three constituents in the natural apitoxin, nothing would have encouraged the person skilled in the art to select these compounds.

In a preferred embodiment of the pharmaceutical composition according to the invention, the said other compound consists of melittin.

As for apamin, the melittin can be naturally purified melittin or synthetized melittin. It must be understood by the person skilled in the art that the expression melittin encompasses any analogue or derivative having the function of melittin according to the invention.

In another preferred embodiment, the pharmaceutical composition according to the invention is characterized by a weight ratio of apamin to melittin is in the range from 0.3:1 to 4:1.

It can be reminded here that the ratio of apamin to melittin in apitoxin or bee venom is in the range of 0.075:1 to 0.06:1 (as apamin represents approximately 3% and melittin represents approximately 40-50% of the constituents of apitoxin). The ratio of apamin to melittin according to the invention is surprising and go beyond the teaching of the prior art.

Nothing in the prior art would have suggested to the person skilled in the art that melittin could have such a “potent” or “synergic” with apamin activity in such low dose.

The ratios of the present invention can be easily calculated by the person skilled in the art regarding the following examples and figures.

In another preferred embodiment, the weight ratio of apamin to melittin is 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1; 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1 or 4:1.

In a still preferred embodiment, the apamin to melittin ratio is selected from 0.3:1, 0.5:1, 0.6:1, 1:1, 1.3:1, 2:1 and 4:1.

In another embodiment of the pharmaceutical composition according to the invention, the said other compound consists of hyaluronidase.

As for apamin, the hyaluronidase can be naturally purified hyaluronidase or synthetized hyaluronidase.

It must be understood by the person skilled in the art that the expression hyaluronidase encompasses any analogue or derivative of hyaluronidase having the function of hyaluronidase according to the invention.

In another preferred embodiment, the pharmaceutical composition according to the invention is characterized by a weight ratio of apamin to hyaluronidase is in the range from 0.003:1 to 0.2:1.

Similarly, it can be reminded here that the ratio of apamin to hyaluronidase in apitoxin or bee venom is in the range of 3:1 to 1.5:1 (as apamin represents approximately 3% and hyaluronidase represents approximately 1-2% of the constituents of apitoxin). The ratio of apamin to hyaluronidase according to the invention is also surprising and go beyond the teaching of the prior art.

Nothing in the prior art would have suggested to the person skilled in the art that hyaluronidase could have such a “potent” or “synergic” with apamin activity in such low dose.

The ratios of the present invention can be easily calculated by the person skilled in the art regarding the following examples and figures.

In another preferred embodiment, the weight ratio of apamin to hyaluronidase is 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.11:1, 0.12:1, 0.13:1, 0; 14:1, 0.15:1, 0.16:1, 0.17:1, 0.18:1, 0.19:1, 0.2:1.

In a still preferred embodiment, the apamin to hyluronidase ratio is selected from 0.003:1, 0.005:1, 0.008:1, 0.01:1, 0.02:1, 0.03:1, 0.05:1, 0.1:1, 0.12:1 and 0.2:1.

In a preferred embodiment of the pharmaceutical composition according to the invention, the said other compound consists of phospholipase A-2. It must be understood by the person skilled in the art that the expression phospholipase A-2 encompasses any analogue or derivative of phospholipase A-2 having the function of phospholipase A-2 according to the invention.

As for apamin, the phospholipase A-2 can be naturally purified phospholipase A-2 or synthetized phospholipase A-2.

In another preferred embodiment, the pharmaceutical composition according to the invention is characterized by a weight ratio of apamin to phospholipase A-2 is in the range from 0.01:1 to 0.2:1.

Similarly, it can be reminded here that the ratio of apamin to phospholipase A-2 in apitoxin or bee venom is in the range of 0.3:1 to 0.25:1 (as apamin represents approximately 3% and phospholipase A-2 represents approximately 10-12% of the constituents of apitoxin). The ratio of apamin to phospholipase A-2 according to the invention is surprising and go beyond the teaching of the prior art.

The ratios of the present invention can be easily calculated by the person skilled in the art regarding the following examples and figures.

In another preferred embodiment, the weight ratio of apamin to phospholipase A-2 is 0.01:1, 0.02:1, 0.03:1, 0.04:1, 0.05:1, 0.06:1, 0.07:1, 0.08:1, 0.09:1, 0.1:1, 0.11:1, 0.12:1, 0.13:1, 0.14:1, 0.15:1, 0.16:1, 0.17:1, 0.18:1, 0.19:1 or 0.2:1.

In a still preferred embodiment, the apamin to phospholipase A-2 ratio is 0.1:1.

In another embodiment, the composition of the invention can comprise in combination with apamin, not only one compound selected from melittin, hyaluronidase or phospholipase A-2; but two compounds consisting of melittin and hyaluronidase, melittin and phospholipase A-2 or hyaluronidase and phospholipase A-2; but also the three compounds melittin and hyaluronidase and phospholipase A-2.

In other words, the pharmaceutical composition according to the invention comprises apamin, and at least another compound, such as without limitation Hyalurinidase, PL-A2, and/or melittin.

Another aspect of the invention relates to the pharmaceutical composition above described for use in the treatment of a degenerative brain disease.

By the expression “degenerative brain disease”, it is intended parkinson's disease but also Alzheimer's disease, etc. . . . .

More particularly, the pharmaceutical composition according to the invention is characterized in that said degenerative brain disease consists of Parkinson disease.

In yet another embodiment, the invention relates to a method of treating a degenerative brain disease by protecting neuronal cell in mammal including human comprising administering an effective amount of a pharmaceutical composition comprising an effective amount of apamin as an active ingredient and an effective amount of at least another compound consisting of a permeability enhancer and/or carrier molecule selected from melittin, hyaluronidase or phospholipase A-2.

The invention will be better illustrated and understood with the following examples and figures wherein:

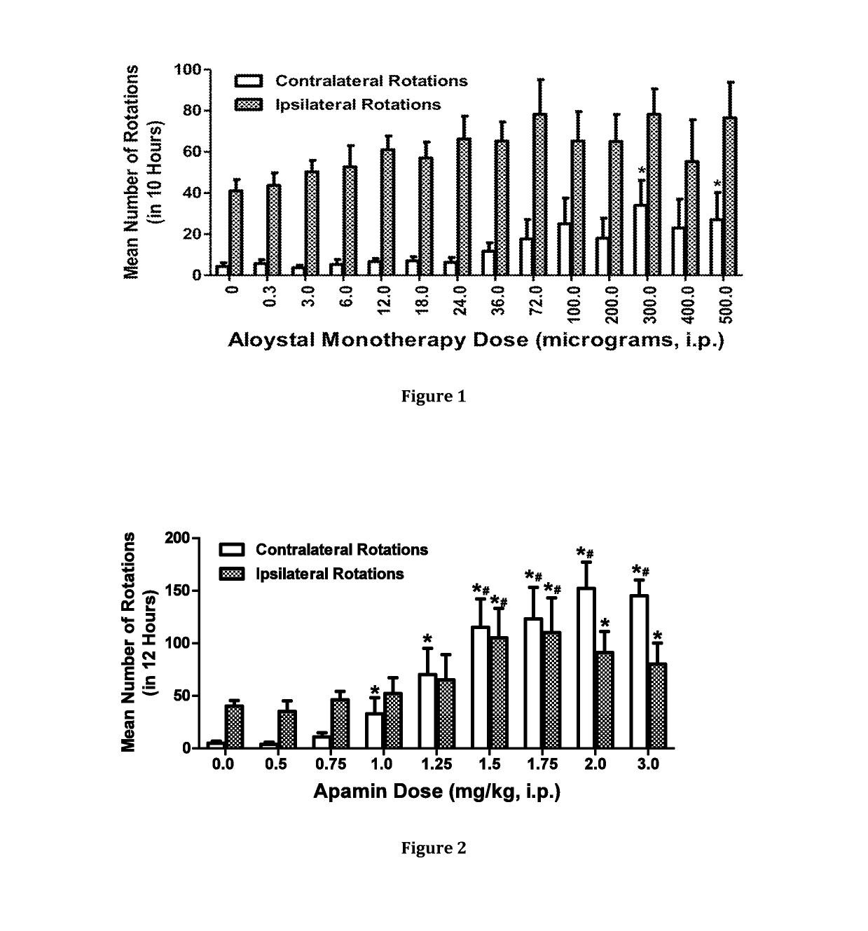

FIG. 1: Apitoxin (Alyostal®; n=3-4 for each dose) induced rotational behaviors in unilaterally 6-OHDA-lesioned animals. Mean total number of rotations over the 10-hour period following apitoxin injection are depicted as means±SE. *p<0.05 compared to 0.0 micrograms (vehicle).

FIG. 2: Apamin (0.0-3.0 mg/kg, i.p.; n=6-10 for each dose) induced rotational behaviors in unilaterally 6-OHDA-lesioned animals. Mean total number of rotations in 12 hours after apamin injection are depicted as means±SE. Rotational behaviors were not assessed beyond 12 hours. *p<0.05 compared to 0.0 mg/kg (Vehicle[PBS]); #p<0.05 compared to 1.0 mg/kg.

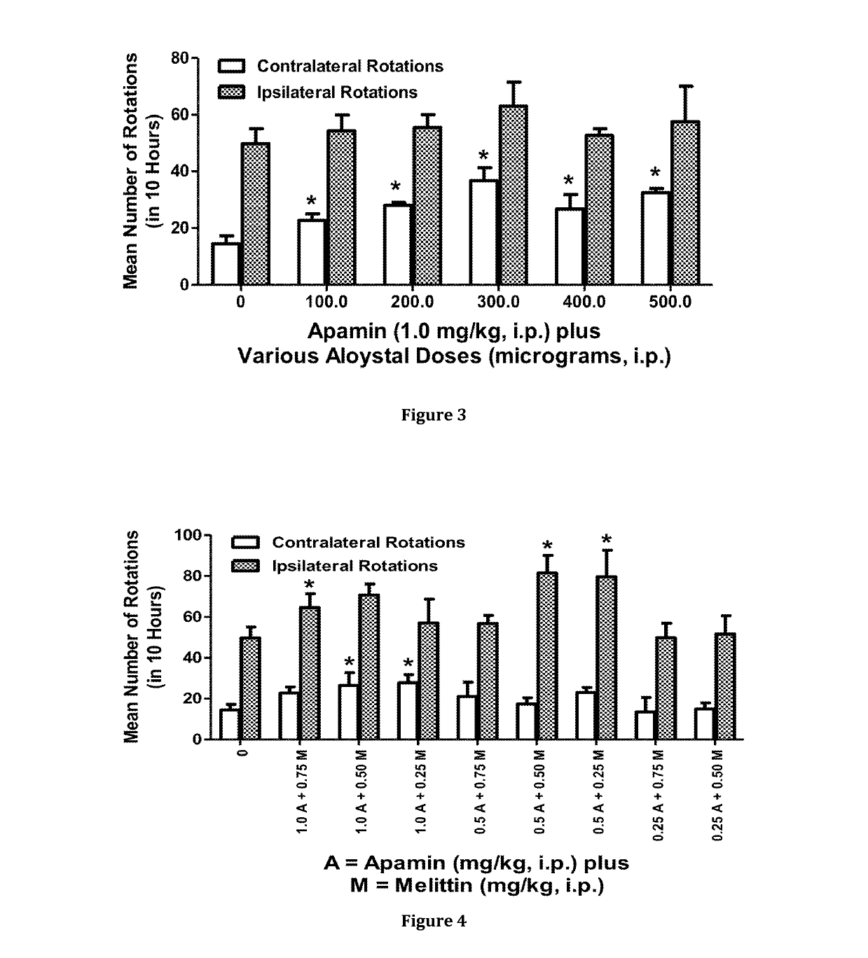

FIG. 3: Apamin (1.0 mg/kg, i.p.) plus Aloystal (100-500 micrograms, i.p.; n=2 to 4 for each dose) increased Apamin-induced rotational behaviors in unilaterally 6-OHDA-lesioned animals. Mean total number of rotations 10 Hours after Apamin+Aloystal co-therapy are depicted as means±SE. Rotational behaviors were not assessed beyond 10 Hours. *p<0.05 compared to Apamin alone group.

FIG. 4: Apamin (0.25-1.0 mg/kg, i.p.) plus melittin (0.25-0.75 mg/kg, i.p.; n=2 to 3 for each dose) increased apamin-induced rotational behaviors in unilaterally 6-OHDA-lesioned animals. Mean total number of rotations 10 Hours after apamin+melittin co-therapy are depicted as means±SE. Rotational behaviors were not assessed beyond 10 Hours. *p<0.05 compared to apamin alone group.

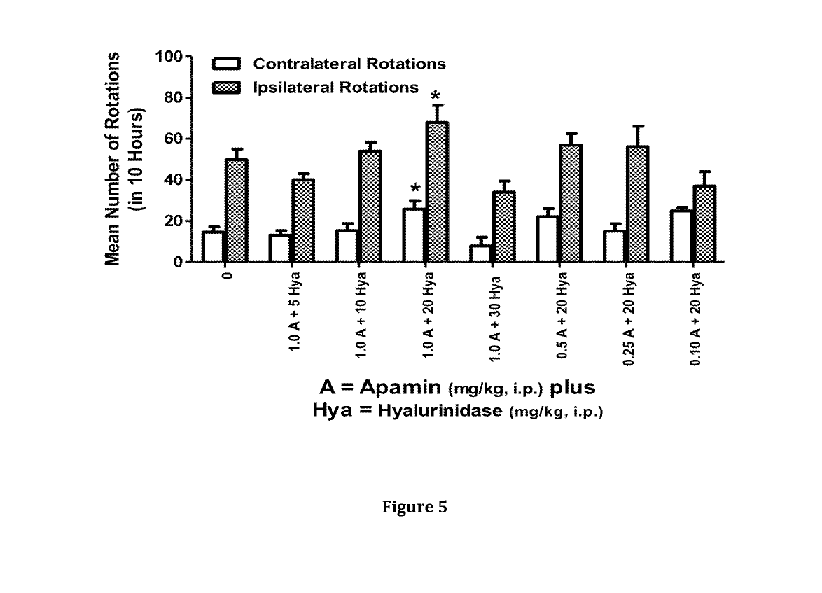

FIG. 5: Apamin (0.10-1.0 mg/kg, i.p.) plus Hyaluronidase (5.0-30 mg/kg, i.p.; n=2 to 3 for each dose) appeared to increase apamin-induced rotational behaviors in unilaterally 6-OHDA-lesioned animals. Mean total number of rotations 10 Hours after apamin+melittin co-therapy are depicted as means±SE. Rotational behaviors were not assessed beyond 10 Hours. *p<0.05 compared to apamin alone group.

The potential antiparkinsonian activity of apitoxin (bee venom; Alyostal®), and certain of its constituents such as apamin, melittin, hyaluronidase, and others were investigated in the standard, unilateral 6-hydroxydopamine (6-OHDA) lesioned rat model, in animals responding to an apomorphine challenge, according to the method previously described with minor modifications (Oh J D, Chartisathian K, Ahmed S M, and Chase T N. (2003) J of Neurosci. Res. 72, 768-780, Oh J D, Chartisathian K, Chase T N and Butcher L L, (2000) Brain Res. 853, 174-185, Oh J D and Chase T N. (2002) Amino Acids 23, 133-139, Oh J D, Del Dotto P, Chase T N. (1997) Neurosci Lett. 228, 5-8, Oh J D, Geller A I, Zhang G, Chase T N. (2003) Br. Res. 971, 18-30, Oh J D, Russell D, Vaughan C L and Chase T N. (1998) Br. Res. 813, 150-159.). Conventional anti-parkinsonian dopamine (DA) agonists, apomorphine, amphetamine, and L-dopa were used as comparator drugs. Control animals received vehicle, phosphate buffered saline (PBS). Rotation (turning) behavior in response to drug challenge was used as a predictor of anti-parkinson activity. All compounds or vehicle were administered either subcutaneously (s.c.) or intraperitonealy (i.p.).

Materials and Methods

The anti-parkinson activity of various test substances was studied as previously described 1-6. Male Sprague-Dawley rats weighing approximately 300 g were housed with free access to food and water. Under Isoflurane gas anesthesia (50 mg/kg, i.p.), the nigrostriatal pathway was unilaterally lesioned by administering 6-OHDA HCl (8 μg in 4 μL of saline with 0.02% ascorbate) into the left medial forebrain bundle (AP −5.0, L 1.3, V 8.0 from the dura mater). Following surgery, rats were challenged with apomorphine (0.1 mg/kg, s.c.), to confirm the lesion of nigro-striatal pathway. Rats that responded to apomorphine were kept for further testing.

Rats received test drugs, comparator drugs, or vehicle on post-surgical Week 3 following 6-OHDA lesioning and subsequently for additional 6 weeks (Week 4-10 post-surgery).

Rotation behavior was measured by placing rats in a rotometer and measuring turning behavior over 5-minute periods (5-min bins). To measure rotation behavior, each animal was placed at least 10 minutes prior to the drug or vehicle administration into a rotometer and turning was measured. Animals then received test drugs, and turning behavior was measured as described above for 4 to 12 hours post-drug administration. The number of turns and duration of rotation exhibited by each animal was measured, as previously described (Oh J D, Chartisathian K, Ahmed S M, and Chase T N. (2003) J of Neurosci. Res. 72, 768-780, Oh J D, Chartisathian K, Chase T N and Butcher L L, (2000) Brain Res. 853, 174-185, Oh J D and Chase T N. (2002) Amino Acids 23, 133-139, Oh J D, Del Dotto P, Chase T N. (1997) Neurosci Lett. 228, 5-8, Oh J D, Geller A I, Zhang G, Chase T N. (2003) Br. Res. 971, 18-30, Oh J D, Russell D, Vaughan C L and Chase T N. (1998) Br. Res. 813, 150-159.).

The following compounds were tested:

TABLE 1

| | |
|---|---|
| Alyostal Bee Venom | Stallergene |
| Apamin | Sigma-Aldrich, #A1289 |
| | (apamin from bee venom) |
| Melittin | Sigma-Aldrich, #M2272 |
| | (Melittin from bee venom approx. 85%) |
| Hyaluronidase | Sigma-Aldrich, #H3506 |
| | (Hyaluronidase type I-S from bovine) |
| Phospholipase A-2 | Sigma-Aldrich, #P9279 |
| | (Phospholipase A-2 from honey bee venom) |
| Tertiapin | Sigma-Aldrich, #T8316 |
| | (Tertiapin-Q trifluoroacetate salt) |
| Secapin | Creative Peptides, #S13001 |
| | (85.6% purity by HPLC) |
| Mast Cell | GenScript, #RP10663 |
| Degranulating Peptide | |
| (MCDP) | |
| Histamine | Sigma-Aldrich, #H7125 |
| | (Histamine free base crystalline) |

Results

Activity of Apitoxin (Alyostal®)

No clear turning (or other) drug-induced motor behaviors were observed with apitoxin at doses lower than 24 μg/rat i.p. (FIG. 1). Clear turning behavior was observed for doses of about 100 to 300 μg per rat and higher. Onset of rotatory behavior was observed within 1 to 2 hours of apitoxin administration. A mixture of rotations both in the direction of the lesion (ipsilateral turning like that produced by amphetamine) and contralateral to the lesion (similar to that produced by apomorphine) was observed. Rotations lasted >10 hours (time points longer than 10 hours were not studied). In contrast to apamin (see below), no other motor behaviors were observed.

Activity of Apamin

Little or no turning behavior above the baseline (i.e. spontaneous rotation) occurred with apamin at doses of <0.75 mg/kg i.p. (FIG. 2). Following the administration of apamin, the onset, duration, magnitude, and morphology of the turning behavior mimicked that of apitoxin. Animals typically manifested alternating bursts of turning in one direction and then in the other, sometimes with intervening periods of relative quiescence. At doses >1 mg/kg i.p., rotatory behavior was observed with an onset of 1 to 2 hours and a duration of usually at least 12 hours (time points longer than 12 hours were not studied). Rotatory behavior was characterized by a mixture of rotations both ipsilateral (like those produced by amphetamine) and contralateral (like those produced by apomorphine) to the lesion. The magnitude of rotational response increased in a dose-related fashion, for up to 2.0 mg/kg dose (FIG. 2).

In addition, at doses of about 1.5 mg/kg, atypical motor behavior began to appear. These involved tremors, dystonic postures, alterations in muscular tone, and locomotor incoordination. The severity of these abnormal motor behaviors was dose-dependent. They began to appear at slightly higher apamin doses than rotatory behaviors and initially were clinically insignificant (at doses of about 0.75 to 1.50 mg/kg). At higher doses (>1.5-3.0 mg/kg), however, they increasingly interfered with normal and rotatory motor behaviors.

Apomorphine or levodopa co-administration at less than standard doses (0.005 mg/kg [s.c.] or 5 mg/kg [i.p.], respectively) appear to mildly potentiate apamin-induced contralateral rotations. On the other hand, amphetamine co-administration at less than the standard dose of 0.05 mg/kg (i.p.) appear to mildly potentiate apamin-induced ipsilateral rotation.

Melittin

Doses of melittin alone from 0.5 to 10 mg/kg i.p. caused a modest, intermittent rotational response with contralateral turning more frequent than ipsilateral turning, especially at the lower doses (Table 2). At highest dose, alternating left and right head turning became prominent and rotational behavior less pronounced.

Melittin (0.5 to 3 mg/kg; FIG. 4) modified the motor response to apamin (0.5 to 1.0 mg/kg) with faster onset of contralateral rotations with mild to absence of atypical motor behaviors that apamin usually induces and that are dose-limiting for apamin; this was especially true for the lower apamin doses.

Hyaluronidase

Given alone, 0.5 to 30 mg/kg i.p. did not induce turning (Table 2).

However, hyaluronidase (5 to 20 mg/kg, i.p.) mildly potentiated ipsilateral turning behavior induced by apamin (0.1 to 1.0 mg/kg; FIG. 5). Most importantly, hyaluronidase decreased the atypical motor behaviors observed with higher doses of apamin given alone (Table 3).

Phospholipase A-2 (PL-A2)

Given alone, 1 mg/kg i.p. produced minimal squirming response, but in doses of approximately 2 and 3 mg/kg i.p, PL-A2 induced ipsilateral rotations (with head turned to opposite direction), starting 1-4 hours after administration and lasting about 4 hours. No atypical motor behaviors were observed.

Given with apamine, PLA-2 accelerated onset of turning behavior and moderately potentiated turning behavior.

Tertiapin

Tertiapin at 1 and 3 mg/kg induced little or no rotational behavior above that of the baseline (i.e. spontaneous rotation).

Secapin

Secapin at a dose of 1 to 20 mg/kg induced little or no rotational behavior above that of the baseline (i.e. spontaneous rotation).

HTGAVLAGV

HTGAVLAGV at a dose of 1 to 10 mg/kg produced little or no turning behavior or other motor activity above that of the baseline (i.e. spontaneous rotation).

The following table 2 illustrates the turning behavior induced by apitoxin and various constituents of apitoxin in unilaterally 6-hydroxydopamine lesioned rats, a model of Parkinson's disease

TABLE 2

| | | | Rotational Response | | |
|---|---|---|---|---|---|
| | Optimally | | (number of | | |
| | Effective | | turns in 12 hours) | | Atypical Motor |
| Drug | Dose Range | | Contralateral | Ipsilateral | Behaviors |
| Vehicle (PBS) | | | 5 | 40 | None |
| Apitoxin | 0.3-500 | μg/rat | 18-27 | 55-78 | None |
| Apamin | 0.75-1.50 | mg/kg | 10-100 | 50-120 | ≧1.5 mg/kg |
| Melittin | 1-3 | mg/kg | 10 | 45 to 50 | None |
| Hyaluronidase | 10 to 30 | mg/kg | 5 | 40 | None |
| Phospholipase A2 | 2 to 3 | mg/kg | 10 | 50 to 60 | None |
| Tertiapin | 1 and 3 | mg/kg | 5 | 40 | None |
| Secapin | 1-20 | mg/kg | 5 | 40 | None |
| HTGAVLAGV | 1-10 | mg/kg | 5 | 40 | None |

The following table 3 illustrates the potentiation of low doses of apamin by various constituents of apitoxin.

TABLE 3

| | | Rotational Response | | |
|---|---|---|---|---|
| | | (number of turns | | |
| | | in 12 hours) | | Atypical Motor |
| Drug | Dose | Contralateral | Ipsilateral | Behaviors |
| Vehicle (PBS) | — | 5 | 40 | None |
| Apamin | 0.5 mg/kg | 5 | 40 | None |
| Apamin | 1 mg/kg | 30 | 50 | Mild |
| Apamin + Apitoxin | Apamin (1 mg/kg) | 40 | 53 to 63 | Decreased vs |
| | Apitoxin (300 μg/kg) | | | Apamin Alone |
| Apamin + Melittin | Apamin 0.5 and 1 mg/kg | 15-30 | 50 to 80 | Mild to None |
| | Melittin 0.25 and 0.5 mg/kg | | | |
| Apamin + | Apamin: 1 mg/kg | 30 | 70 | Decreased |
| Hyaluronidase | Hyaluronidase 20 mg/kg | | | |

As shown in FIG. 3, at all doses of Aloystal tested (at 100-500 micrograms), there was a significant increase in contralateral rotation compared to apamine alone. In addition, the morphology of the rotation was significantly improved in that there was less atypical motor behaviors and more circling behavior (i.e., rotation around the perimeter of the rotational bowl).

As shown in FIG. 4 above, at some of the dose combination tested, there was a significant increase in ipsilateral and/or contralateral rotation compared to apamine alone. The atypical motor behaviors were still present and appeared to be exacerbated when 1.0 mg.kg apamin was used. At lower doses of apamin combined with various doses of melittin, there were no motor side effects present. The most significant finding in apamin plus melittin co-therapy study is that the rotational response shifted to much earlier response (within 30 to 40 minutes after drug injection) compared to apamin alone.

Only at 20 mg/kg Hyalurinidase, there was a significant increase in ipsilateral and contralateral rotation compared to apamine alone. At 10 and 20 mg/kg Hyalurinidase, the apamin-induced atypical motor behaviors were significantly reduced compared to apamin alone. The most significant finding in apamin plus Hyalurinidase co-therapy study is that the motor side effects were significantly reduced with Hyalurinidase compared to apamin alone. However, the increased rotational response appears to be due to the attenuation of motor side effects, and not due to its potentiation effect on apamin-induced response.

1. A pharmaceutical composition comprising:
an effective amount of apamin as an active ingredient and an effective amount of at least another compound including a permeability enhancer and/or a carrier molecule.

2. The pharmaceutical composition according to claim 1, wherein the other compound is melittin.

3. The pharmaceutical composition according to claim 2, wherein the weight ratio of apamin to melittin is in the range from 0.3:1 to 4:1.

4. The pharmaceutical composition according to claim 1, wherein the other compound is hyaluronidase.

5. The pharmaceutical composition according to claim 4, wherein the weight ratio of apamin to hyaluronidase is in the range from 0.02:1 to 0.2:1.

6. The pharmaceutical composition according to claim 1, wherein the other compound is phospholipase A-2.

7. The pharmaceutical composition according to claim 6, wherein the weight ratio of apamin to phospholipase A-2 is in the range from 0.01:1 to 0.2:1.

8. The pharmaceutical composition according to claim 1, for use in the treatment of a degenerative brain disease.

9. The pharmaceutical composition according to claim 8, wherein said degenerative brain disease is Parkinson disease.

10. A method of treating a degenerative brain disease by protecting neuronal cell in mammal including human comprising:
administering an effective amount of a pharmaceutical composition comprising an effective amount of apamin as an active ingredient and an effective amount of at least another compound including a permeability enhancer and/or a carrier molecule selected from melittin, hyaluronidase or phospholipase A-2.
